(19) United States
(12) Patent Application Publication
(10) Pub. No.: US 2025/0073252 A1
(43) Pub. Date: Mar. 6, 2025

(54) NON-PSYCHOACTIVE MULTI-CANNABINOID AND TERPENE-BASED THERAPEUTIC COMPOSITIONS AND METHODS OF THEIR ADMINISTRATION

(71) Applicant: Pebble Global Holdings, Gunter, TX (US)

(72) Inventor: Patrick Moran, Gunter, TX (US)

(21) Appl. No.: 18/681,009

(22) PCT Filed: Aug. 3, 2022
(86) PCT No.: PCT/US2022/039315

§ 371 Date: Feb. 2, 2024

Related U.S. Application Data
(60) Provisional application No. 63/229,044, filed on Aug. 3, 2021.

(51) Int. Cl. A61K 31/00; A61K 31/015; A61K 31/045; A61K 31/715; A61K 36/42; A61K 36/9068; A61K 47/44; A61P 1/08; A61P 25/04; A61P 35/00

(52) U.S. Cl. CPC A61K 31/658; A61K 31/015; A61K 31/045; A61K 31/715; A61K 36/3482; A61K 36/42; A61K 36/9068; A61K 47/44; A61P 1/08; A61P 25/04; A61P 35/00

(57) ABSTRACT

A fixed dose combination formulation drug comprised of three cannabinoids, namely cannabidiol, cannabichromene, and cannabigerol, and of three terpenes, namely alpha-terpinene, bisabolol, and camphene, in an orally available pharmaceutical carrier, for the alleviation of the adverse effects of cancer chemotherapy agents, including adverse effects on the gastrointestinal tract, the chemoreceptor trigger zone, and the peripheral nervous system, and that shows activity adjunctively against ovarian cancer, particularly ovarian cancer that is therapeutically platinum-resistant or platinum-sensitive.

### CROSS-REFERENCE TO RELATED APPLICATIONS

This application is based on, claims priority to, and incorporates by reference in its entirety for all purposes, U.S. Provisional Application Ser. No. 63/229,044, filed Aug. 3, 2021, and entitled, “MULTI-CANNABINOID AND TERPENE-BASED THERAPEUTIC COMPOSITIONS AND METHODS OF THE ADMINISTRATION”.

### FIELD OF THE INVENTION

The present invention relates generally to the treatment of cancers, the treatment of cancer treatment-induced side effects, and to the treatment of neurological disorders. More particularly, the invention relates to: the therapeutic use of fixed dose combinations of cannabinoids, terpenoids and other botanically-derived terpenoid-alkaloids as an adjunctive treatment to one or more current anticancer therapies that have undesirable adverse events and side effects that degrade the patient's quality of life; the production of selective, desired, pharmacologic actions useful in cellular, metabolic, neurological, and other biological system disorders presenting clinically in cancer patients; treatment of chemotherapy-induced peripheral neuropathies; and to treatment of neurological disorders and diseases. The compositions of the invention relate to the scientific fields and subjects of biochemistry, molecular biology, pathology, oncology, pharmacology, pharmacognosy, and botany.

### BACKGROUND OF THE INVENTION

### Cancer.

At this time, there are an estimated 1.9 million new cancer cases diagnosed annually in the United States, and an estimated 609,000 annual cancer deaths. Globally, there are an estimated 17 million new cancer cases being diagnosed annually, a figure that is projected to increase to approximately 27.5 million new cases being diagnosed annually by the year 2040, and there are currently approximately 9.5 million annual cancer deaths.

### Ovarian Cancer.

Ovarian cancer is a good example of a cancer whose incidence, prevalence, lethality, treatment modalities, and treatment side effects are extensively studied in controlled trials, and well documented. In ovarian cancer alone, in the US there are over 230,000 women living with ovarian cancer, and in the year 2022 there will be an estimated 22,000 new annual cases diagnosed, and almost 14,000 ovarian cancer deaths, making ovarian cancer the 5th leading cause of female cancer deaths.

### Ovarian Cancer Chemotherapy.

Regrettably, almost 75% of ovarian cancer patients have advanced/metastatic disease by the time that they are initially diagnosed, typically in Stage III/IV, meaning that chemotherapy must be even more aggressive than in those cases in which the disease is caught early on. Standard of care (SOC) treatments for recurrent ovarian cancer are not curative, and include various combinations of cytotoxic chemotherapies, such as platinum agents including paclitaxel, albumin-bound paclitaxel, docetaxel, gemcitabine, liposomal doxorubicin (Doxil®/Caclyx®), topotecan, bevacizumab, and poly(ADP-ribose) polymerase (PARP) inhibitors. Recently, targeted therapeutics such as PARP-inhibitors (e.g. rucaparib, niraparib, pazopanib, and olaparib) have been approved for patients with deleterious BRCA1/2 mutations (BRCAmut) and as maintenance therapy for patients who initially responded to platinum therapy but who sadly became less responsive or unresponsive to platinum therapy.

### The Increasing Use of Chemotherapy.

The number of chemotherapy treatments, in terms of the number of doses administered, is increasing. This increase is seen to be due to several factors. Firstly, neoadjuvant therapy is increasing in use. Neoadjuvant therapy refers to the use of chemotherapy to reduce a tumor's size prior to a main treatment course. Neoadjuvant chemotherapy (NACT) is often prescribed when a tumor is presently too large to be operable or in cases when vital organs may be impacted by a tumor's growth. NACT alone has increased in use from 17.6% of cases in 2006 to approximately 45% of cases in 2016. Secondly, chemotherapy treatment regimens increasingly rely on a strategy of using combinations of drug agents. For example, targeted therapeutics such as the PARP-inhibitors (e.g. rucaparib, niraparib and olaparib) have recently been approved for patients with deleterious BRCA 1/2 mutations (BRCAmut) and as maintenance therapy for patients who initially responded to platinum therapy, as noted above. Thirdly, as greater success is seen in patient survival rates, follow-on, booster, or additional such treatments are administered to maximize the chance of having attacked all microscopic disease in the patient. And, where the disease shrinks, but does not disappear, chemotherapy will continue life-long for as long as the chemotherapy is tolerated and the disease does not grow.

### Increases in Adverse Events and Degradation of Quality of Life.

Although novel combination treatments, the increasing use of NACT, and the ongoing administration of chemotherapeutic agents will, and do, all have positive effects on patient response, cure rates and remission rates, the converse of this is that the overall volume of adverse events and side effects, throughout the population, continues to grow as well. Virtually all patients suffering from some form of cancer will suffer from a host of illness pathologies, toxicities and problems, both from the disease itself, and from the adverse symptoms of the various medical treatments that are used for fighting cancer. The most common side effects and adverse events (AEs) of standard of care (SOC) therapeutics are nausea and other gastrointestinal effects, and also neuropathies, and these adverse events have a highly significant negative impact on the patient's well-being and quality of life. Such adverse events are furthermore exacerbated in the case of combination therapies, reaching a patient population incidence of over 96%. For example, nausea and gastrointestinal (GI) AEs are seen to occur for single agents in 76% of PARP inhibitors, 100% of platinum agents, 52% of paclitaxel, 69% of gemcitabine, 46% of liposomal doxorubicin, and 63% of topotecan treatment regimens. Neuropathy and neurological symptoms are seen to occur in 18-26% of PARP inhibitors, 10% of paclitaxel, 94% of cisplatinum, and 51% of liposomal doxorubicin treatment regimens. Amplification and exacerbation of side effects can lead to such a loss of quality of life that patients become more likely to limit their dosage intake or to abandon their chemotherapy regimen and compliance altogether, which causes therapeutic failure, and lethal growth of the disease itself.

### The Need for Supportive Care.

The incidence and severity of such symptoms can, in the worst cases, lead to such a degree of exhaustion, depression, and hopelessness that the cancer patient will decide to forego further medical treatment of the cancer itself. The amelioration of the pain of the disease itself, and the adverse events and side effects of the cancer treatment modalities, whether they are chemotherapy or other pharmacological intervention, radiation, or surgery, is an important factor in approaching the supportive care of a cancer patient in a balanced way across all of the quality-of-life challenges facing the patient. There is thus a general need in pharmaceutical therapeutics for treatment regimens that can have a significant counteracting and positive effect on such adverse events and side effects. More generally there is a need for such therapeutics to have improved safety, efficacy, tolerability, and side-effect profiles, manageable duration of action, acceptable manufacturing costs, chemical stability, reliably repeatable batch manufacture, potential for economic scalability as the size of batch outputs increases, affordability, ease of administration, as well as to be readily accessible, lend themselves to packaging, transport and distribution, and to have extended storage life, particularly in remote geographies and in hotter or wetter climates-all being factors in the pursuit of total product feasibility.

### Current Approaches to Supportive Care.

Commonly-prescribed drugs that are employed in attempts to alleviate such cancer chemotherapy side effects include synthetic analogues of tetrahydrocannabinol, e.g. Marinol® (dronabinol), Syndros® (dronabinol), and Cesamet® (nabilone).

### Use of Cannabidiol.

Cannabis sativa extracts have been used in ayurvedic medicine and other medical traditions to treat epilepsy, tetanus, rheumatism, cholera infections, gastrointestinal diseases, and urogenital diseases. These extracts have also gained acceptance and use among cancer patients who are attempting to manage their symptoms. The Cannabis sativa L. plant produces multiple complex chemicals including multiple compounds of the group known as cannabinoids. Cannabidiol (CBD) and hemp oil products have become one of the fastest-growing domestic industries aimed at relieving the pain, nausea and neuropathy associated with chemotherapy, while avoiding or circumventing the delta-9-tetrahydrocannabinol (THC)-associated psychoactive properties of cannabis preparations. Studies have shown that up to 25% of cancer patients have reported consumption of CBD-containing products during their chemotherapy. CBD-containing products are classified by the U.S. Food and Drug Administration as being drugs that require FDA pre-marketing evaluation and approval, but at this time these CBD-containing drug products are also Drug Enforcement Administration Schedule I-classified drugs under the provisions of the Controlled Substances Act. It is therefore illegal to market such CBD products under federal law. There are numerous attempts to market CBD-containing drug and non-drug products under a broad range of unclear and widely differing state laws. For example, although hemp is now lawful under federal law, provided that it has less than 0.3% THC content, it is still illegal to sell hemp products under the laws of the states of Idaho, Nebraska, and South Dakota. The lack of uniform regulation and certification surrounding the composition and manufacturing of these products poses risks for oncologists and other physicians in recommending or prescribing CBD-containing products to their patients. Furthermore, it is risky for the patients to self-medicate with such inadequately standardized products. Medical concerns become even greater due to the potential for drug-drug interactions between CBD and other drugs that the patient is receiving. To date, Epidiolex® is the only FDA-approved prescribed cannabinoid-based drug product (FDA approval is limited to treatment of seizure disorders), but Epidiolex® suffers from the adverse side effect of inducing liver toxicities, particularly when used in combination with other commonly utilized antiepileptic drugs, and Epidiolex's FDA-approved labeling warns prescribers and patients of the possibility of such toxicities. Furthermore, data from Epidiolex studies show that its cannabinoid components can both up-regulate and down-regulate levels of liver enzyme CYP450 isoforms, which are key enzymes in the metabolism and excretion of Epidiolex.

### Multi-Cannabinoid, Multi-Terpenoid Formulations.

Following the hypothesis that a multi-cannabinoid, multi-terpenoid, or multi-terpene formulation (MCTF) will be effective in alleviating chemotherapy-associated side effects in cancer patients undergoing treatment, including nausea, pain, and neuropathy, and that such formulations may furthermore also provide chemotherapy-synergistic anti-tumor efficacy, we have conceived of preferred embodiments of the present invention that comprise drug products which achieve such therapeutic goals, while suppressing the psychoactive properties of the THC component of Cannabis sativa L. extracts. Additional therapeutic indications seen for the most preferred compositions of the present invention include chemotherapeutic-resistant cancers, neurological diseases, e.g. fibromyalgia, anti-aging actions, and oncology & neurology diseases, e.g. chemotherapy-induced peripheral neuropathy (CIPN).

The present invention presents an amelioration modality whose preferred embodiments demonstrate acceptable uniformity of purity, composition, safety, and efficacy, manageable regulatory oversight and control, economic justification of manufacturing and distribution cost, managerial oversight, product development, new business development, and enterprise growth, as well as the primary importance of the potential for expanding patient uptake, utilization, and compliance, and for bringing additional therapeutic indications to ever-increasing patient populations.

### Prior Art Unmet Need

Prior art approaches to the problems inherent in cannabinoid anti-cancer palliative therapies are characterized in that they are inadequately ameliorating, lack standardization, lack uniformity, are of unreliable consistency, lack acceptable purity, are unable to demonstrate absence of contaminant adulteration, have unpredictable effects, lack controlled safety and efficacy tests and trials, have undesirable central nervous system effects, and lack credibility in the eyes of physicians, patients, and government regulatory agencies. Various prior art compositions exist, both approved and prescribed, as well as those that are not in the pharmaceutical industry mainstream marketplace, but their various shortcomings mean that there is an unmet need for a drug composition that must meet multiple essential factors and features in order to constitute a safe, effective, and commercially therapeutic product. Factors and features include being a product that can: effectively bring cancer sufferers relief from the adverse effects and symptoms of the cancer disease itself, as well as the adverse events and symptoms of treatments for cancer; can be standardized to acceptable levels of purity, safety, and efficacy; be free of contamination; have an understandable and viable route to testing, comparison, review, evaluation, and approval by governmental regulatory agencies and the scientific community; deliver predictable, repeatable pharmacologic actions to a patient; gain physician, patient, and third party payor acceptance and trust; be capable of mass production on an industrial scale; and be capable of viable and economically efficient new product development progress and success within an economically acceptable period of development phase activity. The drug compositions, formulations, methods of manufacture, and methods of administration of the present invention meet these needs.

### Objects of the Invention

It is therefore one object of the present invention to produce a cannabinoid-based and terpenoid-based therapeutic drug formulation to alleviate nausea and neuropathy during chemotherapy. Another object of the invention is to produce a formulation that does not promote tumor growth, and preferably has directly anti-tumor effects, does not act as an immune suppressor, and preferably has synergistic properties with standard of care therapeutics. It is another object of the invention to create cannabidiol (CBD)-based and terpene or terpenoid-based human and animal therapeutic products characterized in that they comprise fixed dose combination drug products. Each such combination is defined by having: a plurality of active compound drug product constituents; accurate physico-chemical constituent characterization; and reliable quality standards of crop harvesting, crop processing, pharmaceutical formulating, and manufacturing. It is another object of the invention that such therapeutic products are capable of being tested under statutes and regulations of the U.S. Food and Drug Administration (FDA) Good Laboratory Practice standards (GLP) and being manufactured under current statutes and regulations of the Good Manufacturing Practice standards (cGMP or GMP). Another objective of the invention is to produce a viable product development candidate that can be thoroughly tested, analyzed, characterized, studied, and reported in peer-reviewed academic literature in studies that are repeatable by independent scientific investigators, with the further goal of motivating scientific and medical investigators to design, conduct, and report new knowledge-generating experiments, tests, and trials about CBD pharmacology and toxicology, about its use in therapeutics, and about drug-drug interactions between CBD and other therapeutic drugs.

### SUMMARY OF THE INVENTION

In a preferred embodiment of the invention, a multi-cannabinoid and multi-terpenoid formulation (MCTF) is manufactured and formulated to alleviate the discomfort of the various side effects brought on by treating a cancer patient with one or more cancer chemotherapeutic drugs. This will preferably be a pharmaceutical drug formulation for administration to a patient in need thereof, comprising in fixed dosage one or more compounds that are Cannabis sativa L-derived cannabinoids; more preferably where one or more compounds are cannabinoids selected from the group consisting of cannabinols; and more highly preferred, where one or more compounds are cannabinols selected from the group consisting of cannabidiol (CBD) and the pharmaceutically acceptable salts, esters, solvates, optical isomers, and geometric isomers thereof, present in a molar concentration of from 0.03M to 3.0M; cannabigerol (CBG) and the pharmaceutically acceptable salts, esters, solvates, optical isomers, and geometric isomers thereof, present in a molar concentration of from 0.006M to 0.6M; and cannabichromene (CBG) and the pharmaceutically acceptable salts, esters, solvates, optical isomers, and geometric isomers thereof, present in a molar concentration of from 0.006M to 0.6M; and further comprising one or more terpenes selected from the group consisting of α-terpinene and the pharmaceutically acceptable salts, esters, solvates, optical isomers, and geometric isomers thereof, present in a molar concentration of from 0.022M to 2.2M; bisabolol and the pharmaceutically acceptable salts, esters, solvates, optical isomers, and geometric isomers thereof, present in a molar concentration of from 0.021M to 2.1M; and camphene and the pharmaceutically acceptable salts, esters, solvates, optical isomers, and geometric isomers thereof, present in a molar concentration of from 0.022M to 2.2M.

In a more preferred embodiment of the invention, a multi-cannabinoid and multi-terpenoid formulation (MCTF) is manufactured and formulated to alleviate the discomfort of the various side effects brought on by treating a cancer patient with one or more cancer chemotherapeutic drugs. This will preferably be a pharmaceutical drug formulation for administration to a patient in need thereof, comprising in fixed dosage one or more compounds that are Cannabis sativa L-derived cannabinoids; more preferably where one or more compounds are cannabinoids selected from the group consisting of cannabinols; and more highly preferred, where one or more compounds are cannabinols selected from the group consisting of cannabidiol (CBD) and the pharmaceutically acceptable salts, esters, solvates, optical isomers, and geometric isomers thereof, present in a molar concentration of from 0.06M to 1.5M; cannabigerol (CBG) and the pharmaceutically acceptable salts, esters, solvates, optical isomers, and geometric isomers thereof, present in a molar concentration of from 0.012M to 0.3M; and cannabichromene (CBG) and the pharmaceutically acceptable salts, esters, solvates, optical isomers, and geometric isomers thereof, present in a molar concentration of from 0.012M to 0.3M; and further comprising one or more terpenes selected from the group consisting of α-terpinene and the pharmaceutically acceptable salts, esters, solvates, optical isomers, and geometric isomers thereof, present in a molar concentration of from 0.004M to 1.1M; bisabolol and the pharmaceutically acceptable salts, esters, solvates, optical isomers, and geometric isomers thereof, present in a molar concentration of from 0.04M to 1.1M; and camphene and the pharmaceutically acceptable salts, esters, solvates, optical isomers, and geometric isomers thereof, present in a molar concentration of from 0.04M to 1.1M.

More highly preferred alternative embodiments of the invention include, alternatively, a pharmaceutical formulation wherein one or more of: cannabidiol is present in a molar concentration of 0.3M; where cannabigerol is present in a molar concentration of 0.06M; where cannabichromene is present in a molar concentration of 0.06M; where α-terpinene is present in a molar concentration of 0.22M; where bisabolol is present in a molar concentration of 0.21M; where camphene is present in a molar concentration of 0.22M; where monk fruit extract is present in a concentration ranging from 0.0025% to 0.25% w/w, or more preferably, is present in a concentration of 0.025% w/w; or where ginger essential oil is present in a concentration ranging from 0.059% to 5.9% w/w, or more preferably, 0.59% w/w.

Another embodiment of the invention as claimed is a method of treating an animal or human patient in need thereof, to alleviate the adverse side effects of cancer chemotherapy, cancer chemotherapeutic-induced gastrointestinal side effects, cancer therapeutic-induced peripheral neuropathy, or to be administered to a patient whose cancer, especially ovarian cancer, is chemotherapeutic-resistant or chemotherapeutic-sensitive, comprising the step of administering a pharmacologically effective amount of one or more of the formulations described above.

Another embodiment of the invention as claimed is a method of manufacturing a pharmaceutical formulation comprising one or more cannabinols selected from the group consisting of: cannabidiol and the pharmaceutically acceptable salts, esters, solvates, optical isomers, and geometric isomers thereof, present in a molar concentration of from 0.03M to 3.0M; cannabigerol and the pharmaceutically acceptable salts, esters, solvates, optical isomers, and geometric isomers thereof, present in a molar concentration of from 0.006M to 0.6M; and cannabichromene and the pharmaceutically acceptable salts, esters, solvates, optical isomers, and geometric isomers thereof, present in a molar concentration of from 0.006M to 0.6M; and further comprising one or more terpenes selected from the group consisting of: α-terpinene and the pharmaceutically acceptable salts, esters, solvates, optical isomers, and geometric isomers thereof, present in a molar concentration of from 0.022M to 2.2M; bisabolol and the pharmaceutically acceptable salts, esters, solvates, optical isomers, and geometric isomers thereof, present in a molar concentration of from 0.021M to 2.1M; and camphene and the pharmaceutically acceptable salts, esters, solvates, optical isomers, and geometric isomers thereof, present in a molar concentration of from 0.022M to 2.2M, said method of manufacturing comprising the steps of: (a) combining medium chain triglycerides oil, full spectrum hemp distillate, one or more cannabinols selected from cannabigerol isolate, and cannabichromene distillate into a cooking vessel; (b) heating the combined compositions of step (a) until a temperature of from 140 degrees F. to 200 degrees F. has been reached; (c) cooling the product of step (b) to room temperature; and (d) adding said one or more terpenes and mixing until homogenized. In an alternative method of preparation, the additional step is taken of adding monk fruit extract to said heated mixture, before said cooling begins. In another alternative embodiment of the method of preparation, the additional step of adding ginger essential oil to said cooled mixture is performed.

In a second most preferred embodiment of the invention, a multi-cannabinoid and multi-terpenoid formulation (MCTF) is manufactured, formulated, and administered to alleviate the pain and discomfort of cancer chemotherapy induced peripheral neuropathy.

In a third most preferred embodiment of the invention, a multi-cannabinoid and multi-terpenoid formulation (MCTF) is manufactured, formulated, and administered that is active in directly alleviating and mitigating various forms of cancer in a human or animal patient.

In a fourth most preferred embodiment of the invention, a multi-cannabinoid and multi-terpenoid formulation (MCTF) is manufactured, formulated, and administered that is active in directly alleviating and mitigating undesirable neurological and gastrointestinal symptoms in a human or animal patient.

A most highly preferred embodiment of the invention comprises a formulation whose constituent concentrations are 100 mg/mL CBD, 20 mg/mL CBC, 20 mg/mL CBG, 30 mg/mL alpha-terpinene, 47 mg/mL bisabolol, and 30 mg/mL camphene. A preferred molarity value of the compositions of a preferred formulation comprises α-terpinene 0.22 M, bisabolol 0.21 M, camphene 0.22 M, CBC 0.06 M, CBD 0.3 M, and CBG 0.06 M.

Alternative embodiments of the formulation of the invention include one or more cannabinoids selected from the group consisting of cannabigerol (CBG), cannabidiol (CBD), cannabichromene (CBC), cannabigerivarin (CBGV), cannabidivarin (CBDV), and cannabichromevarin (CBCV).

Alternative embodiments of the formulation of the invention include one or more terpenes selected from the group consisting of alpha-pinene, beta-pinene, camphene, beta-myrcene, D-limonene, 1,8-cineol, alpha-terpinene, gamma-terpinene, cis-beta-terpineol, terpinolene, linalool, fenchol, borneol, alpha-terpineol, alpha-cubebene, ylangene, alpha-copaene, beta-caryophyllene, alpha-bergamotene, alpha-humulene, alloaromadendrene, alpha-amorphene, (−)-lepidozene, beta-selinene, beta-cadinene, alpha-selinene, beta-dihydroagarofuran, alpha-bisabolol, beta-bisabolene, valencene, epizonarene, the selina-dienes, (−)-caryophyliene oxide, guaiol, 8-epi-gammaeudesmol, gamma-eudesmal, agarospiral, alpha-eudesimol, alpha-gurjunene, beta-eudesmaol, alpha-eudesmol, bulnesol, (−)-anymol, delta3-carene, alpha-ocimene, beta-ocimene, trans-alpha-bergamotene, and juniper camphor.

One or more of the compounds cannabidiol, cannabigerol, cannabichromene, α-terpinene, bisabolol, camphene, monk fruit extract, ginger essential oil, or polysaccharide K, which is a protein-bound polysaccharide isolated from the fruitbody of the mushroom Trametes versicolor can be present in any combination of the other compounds. Monk fruit extract, ginger essential oil, and polysaccharide K is widely available commercially at the wholesale and retail levels of distribution.

The invention further comprises methods of treating a patient in need, comprising the steps of administering to the patient a pharmacologically acceptable dose of a pharmaceutical drug formulation comprising one or more cannabinols selected from the group consisting of cannabidiol and the pharmaceutically acceptable salts, esters, solvates, optical isomers, and geometric isomers thereof, present in a molar concentration of from 0.03M to 3.0M; cannabigerol and the pharmaceutically acceptable salts, esters, solvates, optical isomers, and geometric isomers thereof, present in a molar concentration of from 0.006M to 0.6M; and cannabichromene and the pharmaceutically acceptable salts, esters, solvates, optical isomers, and geometric isomers thereof, present in a molar concentration of from 0.006M to 0.6M; and further comprising one or more terpenes selected from the group consisting of: α-terpinene and the pharmaceutically acceptable salts, esters, solvates, optical isomers, and geometric isomers thereof, present in a molar concentration of from 0.022M to 2.2M; bisabolol and the pharmaceutically acceptable salts, esters, solvates, optical isomers, and geometric isomers thereof, present in a molar concentration of from 0.021M to 2.1M; and camphene and the pharmaceutically acceptable salts, esters, solvates, optical isomers, and geometric isomers thereof, present in a molar concentration of from 0.022M to 2.2M. A more preferred method of treating a patient comprises this method, wherein the patient treated for a either cancer therapeutic-induced gastrointestinal adverse effects, cancer therapeutic-induced peripheral neuropathy, drug-resistant cancer, or drug-sensitive cancer, particularly ovarian cancer.

### BRIEF DESCRIPTION OF THE FIGURES

FIG. 1 comprises FIG. 1A, FIG. 1B, and FIG. 1C, and illustrates three charts showing experimentally-derived effective concentrations of monotherapy single agent drugs against selected cancer cell lines.

FIG. 2 is an illustration of a chart showing experimentally-derived effective concentrations of fixed dose combination formulation drug and monotherapy single agent drugs against selected cancer cell lines.

FIG. 3 is an illustration of a layout of four Hill plots illustrating experimental incubations of fixed dose combination formulation drug and monotherapy single agent drugs against selected cancer cell lines.

FIG. 4 comprises FIG. 4A, FIG. 4B, FIG. 4C, and FIG. 4D, and illustrates four charts showing experimentally-derived safety and efficacy data of fixed dose combination formulation drug and monotherapy single agent drug in an in vivo experimental model.

### DETAILED DESCRIPTION OF THE INVENTION

Although the following detailed description contains many specifics for the purposes of illustration, anyone of ordinary skill in the art will appreciate that many variations and alterations to the following details are within the scope of the invention. Accordingly, the following embodiments of the invention are set forth without any loss or diminution of generality to, and without imposing limitations upon, the claimed invention.

As used herein, the words “exemplary” or “illustrative” mean “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art on how to make or use the embodiments of the disclosure and are not intended to limit the scope of the invention, which is defined solely by the claims.

Furthermore, in this detailed description, a person skilled in the art should note that quantitative qualifying terms such as “generally.” “substantially.” “mostly.” and other terms are used, in general, to mean that the referred to object, characteristic, or quality constitutes a majority of the subject of the reference. The meaning of any of these terms is dependent upon the context within which it is used, and the meaning may be expressly modified. Wherever the word “can” is used, it should be taken to be interchangable with the word “may”, both being used in the generic possibility sense.

### Cannabidiol.

Cannabis sativa L. is a plant of the Cannabaceae family, and contains more than 113 known biologically active chemical compounds. The most commonly known compounds are cannabinoids such as delta-9-tetrahydrocannabinol (THC) and cannabidiol (CBD). THC is the component that produces the characteristic euphoria and altered sensory perception associated with marijuana use. CBD is different from marijuana. CBD is but a single compound in the cannabis plant, while marijuana is a type of cannabis plant or plant material that contains many naturally occurring compounds, which includes the two different compounds CBD and THC. CBD itself is a member of the cannabinol group of compounds, which in turn is a member of the cannabinoids group of compounds.

Until December 2018, hemp was included in the Controlled Substances Act (CSA) definition of marijuana and was thus subject to the same restrictions as all other controlled substances in its class. Legislative changes enacted as part of the 2018 Farm Bill (Agriculture Improvement Act of 2018, P.L. 115-334) removed longstanding federal restrictions on the cultivation of hemp. No longer subject to regulation and oversight as a controlled substance by the DEA, hemp production is now subject to regulation and oversight as an agricultural commodity by the U.S. Department of Agriculture (USDA). The 2018 Farm Bill expanded the statutory definition of what constitutes hemp to include “all derivatives, extracts, cannabinoids, isomers, acids, salts, and salts of isomers,” as long as it contains no more than a 0.3% concentration of delta-9 tetrahydrocannabinol (THC); see 7 U.S.C. § 16390. All non-hemp cannabis and cannabis derivatives—including marijuana-derived CBD—are still considered to be marijuana under the CSA and remain regulated by the DEA.

### Cannabidiol Pharmacology and Metabolism.

Cannabidiol is extensively metabolized in liver and gut, primarily by CYP2C19, CYP3A4, and UGT1A7, UGT1A9, and UGT2B7 enzymes. The major circulating metabolites include 7-carboxy-cannabidiol (7-COOH—CBD), which is approximately 40-fold higher than the parent, 7-hydroxy-cannabidiol (7-OH—CBD), which is approximately 38% of the parent based on plasma area under the curve (AUC) of cannabidiol, and 6-hydroxy-cannabidiol (6-OH—CBD), a minor metabolite (<10% of CBD). Cannabidiol and 7-OH—CBD has been found to be equipotent and active. 7-COOH—CBD has been found to be inactive in nonclinical animal models of epilepsy. Cannabidiol is non-psychotropic. Recent evidence shows that the compound counteracts cognitive impairment associated with the use of cannabis. Cannabidiol has little affinity for CB₁ and CB₂ receptors, which are the receptors for THC, and in fact acts as an indirect antagonist of those cannabinoid agonists that do bind to the CB₁ and CB₂ receptors, e.g. THC. Cannabidiol was found to be an antagonist at the putative new cannabinoid receptor, GPR55, a GPC receptor expressed in the brain caudate nucleus and putamen. Cannabidiol has also been shown to act as a 5-HT_{1A} receptor agonist. CBD can interfere with the uptake of adenosine, which plays an important role in biochemical processes, such as energy transfer. It may play a role in promoting sleep and suppressing arousal.

### Terpenes and Terpenoids.

Terpenes are a class of natural products consisting of compounds with the formula (C₅H₈) n. Comprising more than 30,000 compounds, these unsaturated hydrocarbons are produced predominantly by plants, particularly conifers, but also by plants plant of the Cannabaceae family. Terpenes are primary constituents of essential oils and are responsible for the aroma characteristics of cannabis. Together with the cannabinoids, terpenes illustrate synergistic and/or entourage effects. Terpenes are further classified by the number of carbons: monoterpenes (C10), sesquiterpenes (C15), diterpenes (C20), and the like. Although sometimes used interchangeably with “terpenes”, terpenoids (or isoprenoids) are modified terpenes that contain additional functional groups, usually oxygen-containing. Terpenes have been components of traditional medicine, including for the treatment of inflammation. Terpenes are colorless, although impure samples are often yellow. Boiling points scale with molecular size: terpenes, sesquiterpenes, and diterpenes respectively at 110, 160, and 220° C. Being highly non-polar, they are insoluble in water. Being hydrocarbons, they are highly flammable and have low specific gravity (float on water). Terpenoids (mono-, sesqui-, di-, etc.) have similar physical properties but tend to be more polar and hence slightly more soluble in water and somewhat less volatile than their terpene analogues. They are water-soluble solids. They are tactilely light oils considerably less viscous than familiar vegetable oils like corn oil (28 cP), with viscosity ranging from 1 cP (ala water) to 6 cP. Like other hydrocarbons, they are highly flammable. The terms terpenes and terpenoids are used interchangeably in the literature, prior art, in this specification, and the invention as claimed. Terpenes and terpenoids are usually chiral. The terpenes and terpenoids comprising the invention as claimed include racemates, as well as d and/chiral isomers thereof.

A number of terpenes and terpenoids are identifiable in extracts of the Cannabis sativa L. plant. These include, but are not limited to, alpha-pinene, beta-pinene, camphene, beta-myrcene, D-limonene, 1,8-cineol, alpha-terpinene, gamma-terpinene, cis-beta-terpineol, terpinolene, linalool, fenchol, borneol, alpha-terpineol, alpha-cubebene, ylangene, alpha-copaene, beta-caryophyllene, alpha-bergamotene, alpha-humulene, alloaromadendrene, alpha-amorphene, (−)-lepidozene, beta-selinene, beta-cadinene, alpha-selinene, beta-dihydroagarofuran, alpha-bisabolol, beta-bisabolene, valencene, epizonarene, the selina-dienes, (−)-caryophyliene oxide, guaiol, 8-epi-gammaeudesmol, gamma-eudesmal, agarospiral, alpha-eudesimol, alpha-gurjunene, beta-eudesmaol, alpha-eudesmol, bulnesol, (−)-anymol, delta3-carene, alpha-ocimene, beta-ocimene, trans-alpha-bergamotene, and juniper camphor.

Terpenes and cannabinoids can be recovered from the cannabis plant through methods of extraction of botanical oils by methods well known to those of ordinary skill in the art. However, the extraction of the full-spectrum oil consisting, of a full mix of naturally occurring cannabis oils, terpene oil, or cannabinoid oils is presently almost impossible. The most cost-effective way is to selectively separate the terpene oils or cannabinoid oils and include them back into the final desired oil mixture products, including a reconstituted full spectrum oil. Numerous terpene or cannabinoid recovery techniques have been developed by solvent-based or solvent-less techniques. Essential oils are usually hydro-distillated extracts from the trichomes of cannabis. Although most of the constituents remain intact during distillation, a few may undergo chemical changes or are quite often lost due to the nature of the distillation process. The other possible technique is steam distillation by passing dry steam through the inflorescences of the cannabis whereby the terpene oils or cannabinoid oils are volatilized, condensed, and collected. Moreover, the microwave-assisted extraction (MAE) method can enrich bioactive compounds. For example, the MAE treatment using high irradiation power and relatively long extraction times can significantly increase the content of CBD in its essential oil with considerably higher yield when compared with conventional hydro-distillation techniques. In Canada, for instance, the commercial production of the extract is achieved by either solvent extraction such as butane or supercritical fluid (SFE) with the restriction on product purity of no solvent contaminants. The latter technique is known to give superior performance in terpene oil recovery. SFE has recently become a much-preferred method for terpene oil recovery, largely because it allows using lower temperatures, leading to less deterioration of the thermally labile components and is free from organic solvents. A supercritical fluid is a substance brought to a temperature and pressure above its critical points, with no boundary then between the liquid and gas stage. At these points, the fluid is low in viscosity with high diffusion properties to dissolve chemical molecules from the plant matrix. Carbon dioxide (CO₂) is generally used because it is nonflammable, relatively inexpensive, and non-toxic. Large amounts of terpene ingredients are recoverable from this method (e.g., up to 50%, 20% and 10% of caryophyllene, humulene and limonene oils, respectively, can be recovered compared to the conventional methods).

### Terms and Definitions

As used herein the symbols and conventions used in these processes, schemes and examples are consistent with those used in the contemporary scientific literature, for example, the Journal of the American Chemical Society or the Journal of Biological Chemistry. Unless otherwise noted, all starting materials were obtained from commercial suppliers and used without further purification. Specifically, the following abbreviations, acronyms, nomenclature, technical and non-technical term definitions may be used in the examples and throughout the specification.

The phrase “A” or “an” in the context of an entity or moiety as used herein refers to one or more of that entity or moiety, as in for example, “a” compound refers to one or more compounds or at least one compound. As such, the terms “a” (or “an”), “one or more,” and “at least one.” and “and or “can” be used interchangeably. The term “about” has its plain and ordinary meaning of “approximately.” Regarding compound ratios and dosing amounts, and ranges thereof, the qualifier “about” reflects the standard experimental error. In all ranges stated herein, the limits of each range are intended to include as equivalents additional values that are within plus or minus five percent of the figure that states the upper limit or the lower limit of the range. The terms “optional” or “optionally” as used herein means that a subsequently described event or circumstance may, but need not, occur and that the description includes instances where the event or circumstance occurs and instances in which it does not. The term “subject” as used herein, means any species in need of therapy or supplementation, both mammalian animals, and humans. Preferably the subject is a human. The term “preparation” and “compound” or “compounds” and “formulation” or “formulations” is intended to include any of solid, liquid, or gaseous formulations of the active compounds, and one skilled in the art will appreciate that where a compound is an active pharmaceutical ingredient (“API”), it can exist in different preparations depending on the desired dose and pharmacokinetic design parameters. The terms “compositions” and “excipient” and “pharmaceutical excipient” as used herein refer to a compound used to prepare a pharmaceutical composition and is generally safe, non-toxic, and neither biologically nor otherwise undesirable, and includes excipients that are acceptable for veterinary use as well as human pharmaceutical use. The term “dosage” is intended to include either or both solid and liquid formulations of the active compound, and one skilled in the art will appreciate that an active ingredient can exist in different preparations of administration methods, percent of the API, prescribed dose, length of use, time of use, type of indication, desired outcome, and pharmacokinetic design parameters. The term “mixing” or “efficient mixing” as used herein is not limited to the same compounding process; it involves all mixing methods in a manufacturing process. The term “biological system”, as used herein, refers to the interactions of the key elements such as DNA, RNA, proteins, cells, tissues, and organs concerning one another in a subject. The term “Iontophoresis,” as used herein, is a process of transdermal drug delivery by use of a voltage gradient for electromotive drug administration (EMDA) on the skin. Molecules are transported across the stratum corneum by electrophoresis and electroosmosis, and the electric field can also increase the permeability through tissue membranes for diagnostic or therapeutic use. As used herein, “treatment” or “treating” or “therapy” or “therapeutic” or “medicaments” or “prevention” refer to approaches for obtaining beneficial or desired clinical results. Beneficial or desired clinical results include, but are not limited to, prevention, alleviation of symptoms, diminishment of the extent of disease, stabilized state of disease, delay or slowing of disease progression, amelioration or palliation of the disease state, or of the side effects caused by the administration of a therapeutic drug, or of remission of a disease state, whether detectable or undetectable in part or total. “Treatment” can also mean prolonging survival as compared to expected survival if not receiving treatment. “Treatment” is an intervention performed to eliminate or prevent a disease or symptom or develop to eliminate or prevent a disease or alter a medical disorder's pathology in a biological system.

The methods of treatment of the invention comprise administering a safe and effective amount of a fixed dose combination formulation comprised of multiple, or plural, active ingredient compounds in the combination formulation to a patient in need thereof. Individual embodiments of the invention include methods of treating any one of the above-mentioned disorders or conditions by administering a safe and effective amount of a fixed dose combination formulation of the invention to a patient in need thereof.

As used herein, the term “cannabinoids” includes, though not exclusively, the compounds cannabigerol (CBG), cannabidiol (CBD), cannabichromene (CBC), cannabigerivarin (CBGV), cannabidivarin (CBDV), and cannabichromevarin (CBCV).

As used herein, the names annelix or AE2022 are applied to a fixed dose cannabinoid terpenoid formulation originally named in an early clinical setting as Anny's Elixir, consisting of a fixed dose combination of 3 cannabinoids namely cannabidiol, or CBD, cannabichromene, or CBC, and cannabigerol, or CBG, and additionally consisting of 3 terpenes, namely α-terpinene, bisabolol, and camphene.

As used herein, the term α-terpinene means 1-Isopropyl-4-methyl-1,3-cyclohexadiene, p-Mentha-1,3-diene), CAS number 99-86-5, which is a monoterpene found in various plant volatile oils. It is commonly employed as a flavoring additive in foods and beverages, as fragrance in cosmetics, and as scent in household products. It is one of the major components of the essential oil of Melaleuca alternifolia. It reacts with singlet oxygen to form the endoperoxide, ascaridole along with p-cymene and hydroperoxides.

The term bisabolol, or more formally α-(−)-bisabolol or also known as levomenol, is a natural monocyclic sesquiterpene alcohol, CAS number 23089-26-1, IUPAC name (2S)-6-Methyl-2-[(1S)-4-methylcyclohex-3-en-1-yl]hept-5-en-2-ol. It is a colorless viscous oil that is the primary constituent of the essential oil from German chamomile (Matricaria recutita) and Myoporum crassifolium.

The term camphene means 2,2-Dimethyl-3-methyllidenebicyclo[2.2.1]heptane, CAS number 79-92-5.

As used herein, “treat” or “treating” in reference to a disorder means: (1) to ameliorate or prevent the disorder or one or more of the biological manifestations of the disorder; (2) to interfere with (a) one or more points in the biological cascade that leads to or is responsible for the disorder or (b) one or more of the biological manifestations of the disorder; (3) to alleviate one or more of the symptoms or effects associated with the disorder; or (4) to slow the progression of the disorder or one or more of the biological manifestations of the disorder.

As indicated above, “treatment” of a disorder includes prevention of the disorder. The skilled artisan will appreciate that “prevention” is not an absolute term. In medicine, “prevention” is understood to refer to the prophylactic administration of a drug to substantially diminish the likelihood or severity of a disorder or biological manifestation thereof, or to delay the onset of such disorder or biological manifestation thereof.

As used herein, “safe and effective amount” in reference to the disclosed and claimed drug product, which is a fixed dose combination formulation, or to any of its constituent active compositions or compounds that comprise the formulation, means an amount sufficient to treat the patient's condition but low enough to avoid serious side effects (at a reasonable benefit/risk ratio) within the scope of sound medical judgment. A safe and effective amount of the drug product will vary with the particular constituent composition or compound chosen (e.g. considerations of the potency, efficacy, and half-life of the composition or compound); the route of administration chosen; the disorder being treated; the severity of the disorder being treated; the age, size, weight, and physical condition of the patient being treated; the medical history of the patient to be treated; the duration of the treatment; the nature of a concurrent therapy, if any; the desired therapeutic effect; and similar or like factors, but can nevertheless be routinely determined by the skilled artisan.

Full Spectrum Hemp Distillate (FSO) is the distillation product that is characterized in having all of the cannabinoids found in whole hemp plant present, and can be prepared in compliance with current Good Manufacturing Practice (cGMP).

CBD Isolate is cannabidiol in isolated, pure form, unlike CBD distillate, which contains an array of cannabinoids, terpenes, vitamins, and fatty acids.

As used herein, “patient” refers to a human (including adults and children) or to an animal. In one preferred embodiment, “patient” refers to a human.

The fixed dose combination formulations of the invention may be administered by any suitable route of administration, including both systemic administration and topical administration. In the most highly preferred embodiment of the invention, administration is via the oral route. Systemic administration includes oral administration, parenteral administration, transdermal administration and rectal administration. Parenteral administration refers to routes of administration other than enteral or transdermal, and is typically by injection or infusion. Parenteral administration includes intravenous, intramuscular, and subcutaneous injection or infusion. Topical administration includes application to the skin as well as nasal, intraocular, otic, intravaginal, inhaled and intranasal administration. Inhalation refers to administration into the patient's lungs whether inhaled through the mouth or through the nasal passages.

The fixed dose combination formulations may be administered once or according to a dosing regimen wherein a number of doses are administered at varying intervals of time for a given period of time. For example, doses may be administered one, two, three, or four times per day. In one embodiment, a dose is administered once per day. In a further embodiment, a dose is administered twice per day. Doses may be administered until the desired therapeutic effect is achieved or indefinitely to maintain the desired therapeutic effect. Suitable dosing regimens for the fixed dose formulation drug product depend on the pharmacokinetic properties of the constituent compositions, such as absorption, distribution, and half-life, which can be determined by the skilled artisan. In addition, suitable dosing regimens, including the duration such regimens are administered, for a polymorph, isomer, or salt of the invention depend on the disorder being treated, the severity of the disorder being treated, the age and physical condition of the patient being treated, the medical history of the patient to be treated, the nature of concurrent therapy, if any, the desired therapeutic effect, and like factors that are within the knowledge and expertise of the skilled artisan. It will be further understood by such skilled artisans that suitable dosing regimens may require adjustment given an individual patient's response to the dosing regimen or over time as individual patient needs change or as liver and or kidney functional capacity change.

Typical daily dosages may vary depending upon the particular route of administration chosen. Typical daily dosages of the individual active constituent compositions that comprise the fixed dose formulation, via oral administration, which is the most preferred embodiment of the invention, range from 0.001 mg to 50 mg per kg of total body weight, for example more preferably from 1 mg to 10 mg per kg of total body weight. Daily dosages of the individual active constituent compositions that comprise the fixed dose formulation, for oral administration, may be from 0.5 mg to 2 g per patient, for example such as 10 mg to 1 g per patient. The pharmaceutical formulations of the invention preferably comprise from two to eight constituent compositions, in any combination thereof, fixed in their relative ratios of weight or volume to each other.

As used herein. “pharmaceutically acceptable excipient” means a pharmaceutically acceptable material, composition or vehicle involved in giving form or consistency to the drug product pharmaceutical formulation. Each excipient must be compatible with the other ingredients of the pharmaceutical formulation when they are in, for example, a blending stage of manufacture and being commingled, such that physicochemical interactions are avoided that would substantially reduce the efficacy of one or more of the constituent compositions of the invention when the formulation drug product is administered to a patient, as well as to avoid interactions that would result in pharmaceutical drug product formulations being produced that are not pharmaceutically acceptable. In addition, each excipient must of course be pharmaceutically acceptable, i.e., of sufficiently high purity and in conformity with the applicable monograph of an official compendium.

The preferred constituent compositions of the invention and the pharmaceutically acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as nasal sprays, aerosols, solutions, and dry powders; and (6) topical administration such as creams, ointments, lotions, solutions, pastes, sprays, foams, and gels.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting of a given active constituent composition of the invention once administered to the patient from one organ, or portion of the body, to another organ, or portion of the body. Certain pharmaceutically acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, humectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other excipients are present in the formulation.

Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company) or The Theory and Practice of Industrial Pharmacy (Lea & Febiger), the disclosures of which are incorporated herein by reference.

Accordingly, in another aspect the invention is directed to a formulation process or manufacturing process for the preparation of a pharmaceutical drug product that is a fixed dose combination comprised of multiple active constituent compositions and one or more pharmaceutically acceptable excipients which comprises mixing the ingredients. A pharmaceutical composition comprising a polymorph, isomer, or salt of the invention may be prepared by, for example, admixture at ambient temperature and atmospheric pressure.

In one preferred embodiment, the invention may be formulated for oral administration. In another embodiment, the polymorph or salt of the invention may be formulated for inhaled administration. In a further embodiment, the polymorph or salt of the invention may be formulated for intranasal administration.

In one aspect, the invention is directed to a solid oral dosage form such as a tablet or capsule comprising safe and effective amounts of the pharmacologically active constituents of the fixed dose combination formulation, and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (again e.g. corn starch, potato starch, and pre-gelatinized starch), gelatin, acacia, sodium alginate, alginic acid, tragacanth, guar gum, povidone, and cellulose or a cellulose derivative (e.g. microcrystalline cellulose). The oral solid dosage form of the preferred embodiment of the invention may further comprise a disintegrant. Suitable disintegrants include crospovidone, sodium starch glycolate, crosscarmellose, alginic acid, and sodium carboxymethyl cellulose. The oral solid dosage form may further comprise a lubricant. Suitable lubricants include stearic acid, magnesium stearate, calcium stearate, and talc.

Where appropriate, dosage unit formulations for oral administration can be microencapsulated. Microencapsulation can be used to keep constituent compositions segregated from one another until the gastrointestinal tract breakdown and absorption process has had an opportunity to reach and digest the microcapsules individually. Alternatively microencapsulation can be used to generate discrete populations of microcapsules that will break down at differing time intervals following ingestion, or in different locations along the gastrointestinal tract. This type of breakdown profile can readily be achieved by one of ordinary skill in the art by for example selectively coating or embedding particulate material(s) in polymers, wax or the like.

The pharmaceutically acceptable salts of the compounds used in the compositions of the invention include the conventional non-toxic salts or the quarternary ammonium salts of said compounds formed, e.g., from non-toxic inorganic or organic acids, and for example, such conventional non-toxic salts include those derived from inorganic acids such as hydrochloric. hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Generally, the salts are prepared by reacting the free base or acid with stoichiometric amounts or with an excess of the desired salt-forming inorganic or organic acid or bases in a suitable solvent or various combinations of solvents. The pharmaceutically acceptable salts are also readily prepared by conventional procedures such as treating an acid with an appropriate amount of a base, such as an alkaline or alkaline earth metal hydroxide e.g. sodium, potassium, lithium, calcium, or magnesium, or an organic base such as an amine, e.g., dibenzyl ethylenediamine, trimethylamine, piperidine, pyrrolidine, benzylamine and the like, or a quaternary ammonium hydroxide such as tetramethylammonium hydroxide and the like.

Suspensions and solutions comprising a polymorph, isomer, or salt of the invention may also be administered to a patient via the oral route or the nasal route via a suitable spray and pump. The solvent or suspension agent utilized for ingestion or nebulization may be any pharmaceutically-acceptable liquid such as water, aqueous saline, alcohols or glycols, e.g., ethanol, isopropylalcohol, glycerol, propylene glycol, polyethylene glycol, etc. or mixtures thereof. Saline solutions utilize salts which display little or no pharmacological activity after administration. Both organic salts, such as alkali metal or ammonium halogen salts, e.g., sodium chloride, potassium chloride or organic salts, such as potassium, sodium and ammonium salts or organic acids, e.g., ascorbic acid, citric acid, acetic acid, tartaric acid, etc. may be used for this purpose.

Other pharmaceutically acceptable excipients may be added to the suspension or solution. Constituent compositions of the invention may be stabilized by the addition of an inorganic acid, e.g., hydrochloric acid, nitric acid, sulphuric acid and/or phosphoric acid; an organic acid, e.g., ascorbic acid, citric acid, acetic acid, and tartaric acid, etc., a complexing agent such as EDTA or citric acid and salts thereof; or an antioxidant such as vitamin E or ascorbic acid. These may be used alone or together to stabilize a given constituent composition of the invention. Preservatives may be added such as benzalkonium chloride or benzoic acid and salts thereof. A surfactant may be added particularly to improve the physical stability of suspensions. These include lecithin, disodium dioctylsulfosuccinate, oleic acid and sorbitan esters.

In a further aspect, the invention is directed to a dosage form adapted for intranasal administration. Formulations for administration to the nose may include pressurized aerosol formulations and aqueous formulations administered to the nose by pressurized pump. Formulations which are non-pressurized and adapted to be administered topically to the nasal cavity are of particular interest. Suitable formulations contain water as the diluent or carrier for this purpose. Aqueous formulations for administration to the lung or nose may be provided with conventional excipients such as buffering agents, tonicity modifying agents and the like. Aqueous formulations may also be administered to the nose by nebulization.

Pharmaceutical compositions adapted for transdermal administration may be presented as discrete patches intended to remain in intimate contact with the epidermis of the patient for a prolonged period of time. For example, the active ingredient may be delivered from the patch by iontophoresis as generally described in Pharmaceutical Research, 3(6), 318 (1986).

Pharmaceutical compositions adapted for topical administration may be formulated as ointments, creams, suspensions, lotions, powders, solutions, pastes, gels, sprays, aerosols or oils. Ointments, creams and gels, may, for example, be formulated with an aqueous or oily base with the addition of suitable thickening and/or gelling agent and/or solvents. Such bases may thus, for example, include water and/or an oil such as liquid paraffin or a vegetable oil such as arachis oil or castor oil, or a solvent such as polyethylene glycol. Thickening agents and gelling agents which may be used according to the nature of the base include soft paraffin, aluminum stearate, cetostearyl alcohol, polyethylene glycols, woolfat, beeswax, carboxypolymethylene and cellulose derivatives, and/or glyceryl monostearate and/or non-ionic emulsifying agents. Lotions may be formulated with an aqueous or oily base and will in general also contain one or more emulsifying agents, stabilising agents, dispersing agents, suspending agents or thickening agents. Topical preparations may be administered by one or more applications per day to the affected area; over skin areas occlusive dressings may advantageously be used. Continuous or prolonged delivery may be achieved by an adhesive reservoir system.

It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates to optimize the activity and/or stability and/or physical characteristics, such as solubility, of the therapeutic ingredient. It will be clear also that, where appropriate, the therapeutic ingredients may be used in optically pure form.

When the name of a commercial supplier is given after the name of a compound or a reagent, for instance “compound X (Aldrich)” or “compound X/Aldrich”, this means that compound X is obtainable from a commercial supplier, such as the commercial supplier named. If not referenced herein the compound or reagent can be purchased from a standard supplier such as Sigma Aldrich, Lancaster, Fluorochem, TCI, and the like.

### Prior Art Problem of the Synergistic Entourage Effect.

The claimed method of manufacture of the drug product invention as claimed has overcome a problem in the prior art, which is the failure to uniformly achieve and maintain a maximally synergistic medicinal effect of the combined terpene and cannabinoid compounds/ingredients of a fixed dose formulation in presenting a therapeutic entourage to the patient's system. The entourage effect is explained as increased activity of an active compound with an inactive one. There is a demonstrated entourage effect between cannabinoids and terpenes, but prior art methodologies of combining members of these two families of compounds have resulted in diminution or loss of the effect, owing either to the extraction, purification, and recombination methods used, or to the molar ratios being used. The synergistic therapeutic entourage effect of the invention can consistently maintain pharmaceutical variance standards within a range of 10%, which is a superior entourage result over prior art methods of preparation for a formulation that delivers multiple compounds in the families of cannabinoids and terpenes.

### Cancers and Their Treatment.

The next major goal and feature of the compositions of the invention is directed to addressing the treatment of cancer. The activity of Pgp ATPase inhibition has been demonstrated which was dependent on the duration of exposure resulting in reduction of Pgp expression in chronic exposure and an increase of Pgp expression during acute exposure. Cytochrome P450 inhibition prevented conversion of tamoxifen, and interference with gefitinib, imatinib, cyclophosphamide, lapatinib, anastrozole and dasatinib metabolism have been observed. Such observations suggest the application of cannabinoid drug formulations directly against cancer. Cancers include Actinic keratoses (Aks), Acute Lymphoblastic Leukemia (ALL), Acute Myeloid Leukemia (AML), Adrenal gland tumors, Anal cancer, Basal cell carcinoma, Bile duct cancer, Bladder cancer, Blood cancers, Bone cancer, Bowel cancer, Brain tumors, Breast cancer, Cancer of Unknown Primary (CUP), Cancer metastasis to bone, Cancer metastasis to brain, Cancer metastasis to liver, Cancer metastasis to lung, Carcinoid, Cervical cancer, Children's cancers, Chronic Lymphocytic Leukemia (CLL), Chronic Myeloid Leukemia (CML), Colorectal cancer, Ear cancer, Endometrial cancer, Eye cancer, Follicular dendritic cell sarcoma, Gallbladder cancer, Gastric cancer, Gastro-esophageal junction cancers, Germ cell tumors, Gestational Trophoblastic Disease (GTD), Hairy cell leukemia, Head and neck cancer, Hodgkin's lymphoma, Kaposi's sarcoma, Kidney cancer, Large bowel and rectal neuroendocrine tumors, Laryngeal cancer, Leukemia, Linitis plastica of the stomach, Liver cancer, Lung cancer, Lung Neuroendocrine Tumors (NETs), Lymphoma, Malignant schwannoma, Mediastinal germ cell tumors, Melanoma skin cancer, Men's cancer, Merkel cell skin cancer, Mesothelioma, Molar pregnancy, Mouth and oropharyngeal cancer, Myeloma, Nasal and paranasal sinus cancer, Nasopharyngeal cancer, Neuroblastoma, Neuroendocrine Tumors, Neuroendocrine Tumors of the pancreas, Non-Hodgkin lymphoma, Non-Hodgkin lymphoma in children, Esophageal cancer, Ovarian cancer, Pancreatic cancer, Penile cancer, Persistent trophoblastic disease and choriocarcinoma, Phacochromocytoma, Prostate cancer, Pseudomyxoma peritonei, Rare cancers, Rectal cancer, Retinoblastoma, Salivary gland cancer, Secondary cancer, Signet cell cancer, Skin cancer, Small bowel cancer, Small bowel Neuroendocrine Tumors (NETs), Soft tissue sarcoma, Stomach cancer, Stomach Neuroendocrine Tumors (NETs), Squamous cell carcinoma, Testicular cancer, Thymus gland tumors, Thyroid cancer, Tongue cancer, Tonsil cancer, Tumors of the adrenal gland, Unknown primary cancer, Uterine cancer, Vaginal cancer, Vulvar cancer, Wilms' tumors, Womb cancer, and Women's cancers (gynecological cancer). Pre-clinical studies using pure CBD extracts in various cancer models have shown direct anti-tumor effects at low micromolar levels and synergy with chemotherapeutics in vitro including cisplatinum, carmustine, temozolomide, doxorubicin, paclitaxel, vincristine, and bortezomib.

### EXAMPLES

The invention will now be illustrated by way of the following non-limiting examples. These examples are not intended to limit the scope of the present invention, but rather to provide guidance to the skilled artisan, without undue experimentation, to prepare and use the compositions, their polymorphs, salts, esters, optical isomers, geometric isomers, hydrates, and solvates, in fixed dose combination formulation drug products. While particular embodiments of the present invention are described, the skilled artisan will appreciate that various changes and modifications can be made without departing from the spirit and scope of the invention.

### Example 1. Preparation of an MCT Formulation

Starting ingredients: fractionated coconut oil (also known as medium chain triglycerides or MCT), full spectrum hemp distillate (FSO), which provides the source of cannabiciol (CBD) of the formulation, CBG isolate (CBG), CBC distillate (CBC), alpha terpinene, camphene, alpha bisalol, ginger essential oil (food grade), and monk fruit extract, all of which are readily available for purchase from commercial sources. Equipment: 5-gallon NSF-grade stainless steel cooking vessel, stainless steel mixing spoon, Ohaus Defender 3000 30 kg capacity bench scale, Ohaus Scout 6200 gram capacity bench scale, Nuwave induction cooktop burner, NIST-calibrated thermometer, and a high speed shear mixer. Procedure: CBD, CBC, and CBG are preferably isolated botanical extracts from the flowers of the Cannabis sativa L plant. Alpha terpinene is preferably an isolated botanical extract from the Syncarpia glomulifer plant Camphene is preferably isolated from botanical camphor oil. The following ingredient amounts were weighed out, where the weight was determined and expressed as w/w as follows: alpha terpinene—3% w/w; camphene—3% w/w; alpha bisabolol—5% w/w; ginger essential oil (food grade)—0.59% w/w; and monk fruit extract—0.025% w/w. An initial determination of the amount of each ingredient was calculated by assessing the number of final product dosage units that was needed from the production process. The average density of the MCT (fractionated coconut oil) was calculated to be 0.94 g/ml, reflecting the target ingredient amounts of 100 mg/mL CBD, 20 mg/mL CBC, and 20 mg/mL CBG, 30 mg/mL alpha-terpinene, 47 mg/mL bisabolol, and 30 mg/mL camphene. Molar concentrations of the final product were prepared for, and produced, α-terpinene 0.22 M, bisabolol 0.21 M, camphene 0.22 M, CBC 0.06 M, CBD 0.3 M, and CBG 0.06 M. The average density of the MCT. 0.94 g/ml, was assumed as an average density of the final product. This average density was multiplied by the final mass of product to be produced. The volume or weight amount of full spectrum distillate (FSO) needed was determined by determining the percentage present of cannabidiol (CBD) in the FSO through ordinary qualitative and quantitative analytical methods well known to those of ordinary skill in the art, and then documented on a Certificate of Analysis (COA) as provided by the commercial supplier. The COA included: (a) a listing of tests performed by the analytical laboratory, the test date(s), and the test results, and a certification of the accuracy of each of the foregoing; and (b) a cross-reference to the related Certificate of Compliance. Determination of the percentage, and therefore the concentration, of CBD in the distillate, enabled the determination of the amount of FSO distillate needed in the formulation according to the equation D=(T*CT)/P_{D}, where D=the total amount of distillate needed, in grams, T=the total number of tinctures or capsules or tablets (the dosage form) needed, CT=the target final concentration of CBD in the dosage form, and P_{D}=the percentage of CBD contained in the distillate, obtained from a contract quantitative analytical laboratory. Once the amount of FSO distillate needed for the batch had been calculated, then the amount of CBG isolate and CBC distillate needed was calculated by determining the amounts of CBG and CBC present in the FSO distillate, then calculating the amount of CBG isolate and CBC distillate that was needed to bring final product up to target CBG and CBC levels. Concentration of CBG in the CBG isolate and concentration of CBC in the CBC distillate was determined by reference to the COA for each that was provided by their respective commercial sources. Once the supplementary amounts of CBG and CBC were determined for addition to the FSO distillate, the amount of the MCT needed for the final output was weighed in a stainless-steel cooking vessel, to which were added the calculated amounts of the FSO distillate, the CBG isolate, and the CBC distillate. The mixture was heated to 170° F. (77° C.) and maintained, taking care to ensure that during the warming phase, all of the cannabinoids were being thoroughly mixed into and throughout the MCT. Once a homogeneous solution was observed, the monk fruit extract was added and thoroughly mixed in a high shear mixer for five minutes (the solution was mixed for ten minutes in another batch run). The mixed solution was allowed to cool to room temperature at which point the terpenes and ginger essential oil was added to the vessel and mixed until thoroughly homogenized. The resulting product batch was analyzed for the target cannabinoid and terpene amounts and to ensure freedom from toxic heavy metals or agricultural pesticide residues. Upon confirmation of the potency and safety of the batch, it was divided into aliquots, bottled, packaged, and stored under cool, dry conditions.

### Evaluation of Multi-Cannabinoid Formulations in Humans and Animals.

Pre-clinical drug product development requires successful outcomes in animal model efficacy studies, and in animal model toxicity studies prior to seeking FDA government regulatory approval for conducting more advanced studies in human volunteer clinical trials. Multiple in-house open label case studies of a most preferred embodiment of the invention, annelix, the product of Example 1 above, across a wide array of indications in both animals (canines) and humans for indications including pancreatic cancer, osteosarcoma, fibromyalgia, degenerative arthritis, adult autism, and epileptic seizures, and no adverse side effects have been observed or reported.

### Example 2. Female Pancreatic Cancer

A 63 year old female pancreatic cancer patient reported complete elimination of post-chemotherapy nausea and neuropathy, along with a significant decrease in the severity of inflammatory pain.

### Example 3. Osteosarcoma in a Great Dane Canine

The owner of a Great Dane diagnosed with osteosarcoma reported that veterinarian X-rays showed an approximately 50% reduction in bone tumor mass.

### Example 4. Female Fibromyalgia

A 46 year old female fibromyalgia patient reported via the Likert Scale (1-10) that daily pain improved from an assessment of eight (8) to an assessment of three (3).

### Example 5. Comparison of Cannabinoid Single Agent to Combination Products

An in vitro study was conducted, whose purpose was to determine the anti-cancer activity of cannabinoids as single agents and to compare their activities with a preferred embodiment of the invention, a cannabinoid/terpene formulation comprising three (3) cannabinoids, namely cannabidiol, cannabichromene, and cannabigerol, and additionally comprising the terpenoids alpha terpinene, bisabolol and camphene (annelix). Single agent terpenoids had earlier demonstrated anti-oxidant and anti-inflammatory properties, but showed only marginal or no effects against various cancer cell lines, e.g. (IC₅₀ values>50 uM) for α-bisabolol against ALL 53, 73 μM (normal endothelial cells 5 μM), camphene against B16-F10 melanoma, 529 UM and γ-terpinene against leukemia (P388), liver cancer (HepG2), skin melanoma (B16-F10), and myelogenous leukemia (K562) 60-180 μM. Synergistic relationships for combinations of terpenoids and cannabinoids have been reported Russo, E. B. and J. Marcu, Chapter Three-Cannabis Pharmacology: The Usual Suspects and a Few Promising Leads, in Advances in Pharmacology, D. Kendall and S. P. H. Alexander, Editors. 2017, Academic Press. p. 67-134. This observed synergy was strongly dependent on the ratio and origin of terpenoids, as shown in triple negative breast cancer models. In addition, many cannabinoids and terpenoids are immune suppressors as reported for α-bisabolol, which activates TGF-β to promote the differentiation of macrophages into the M2 phenotype (tumor promoting) and regulatory T cells. Ovarian cancer cell lines tested included A2780ip2, A2780CP20, HeyA8, HeyA8-MDR, SKOV3 and OVCAR3 and were maintained in RPMI 1640 supplemented with 10-15% fetal bovine serum and 0.1% gentamicin sulfate (Gemini Bioproducts, Calabasas, CA, USA). OVCAR5 cells were maintained in Dulbecco's modified Eagle's medium with 10% fetal bovine serum and 0.1% gentamicin sulfate. ID8 murine ovarian cancer cells were maintained in Dulbecco modified Eagle medium containing 10% fetal bovine serum (FBS), 1× insulin-transferrin-selenium (Thermo Fisher Scientific, Waltham, MA), and 0.5% gentamicin. All above cells were cultured at 37° C. using a 5% CO₂ incubator. All in vitro experiments were performed with 70-80% confluent cultures and a passage number below.

### Cell Viability Assays.

### Example 6

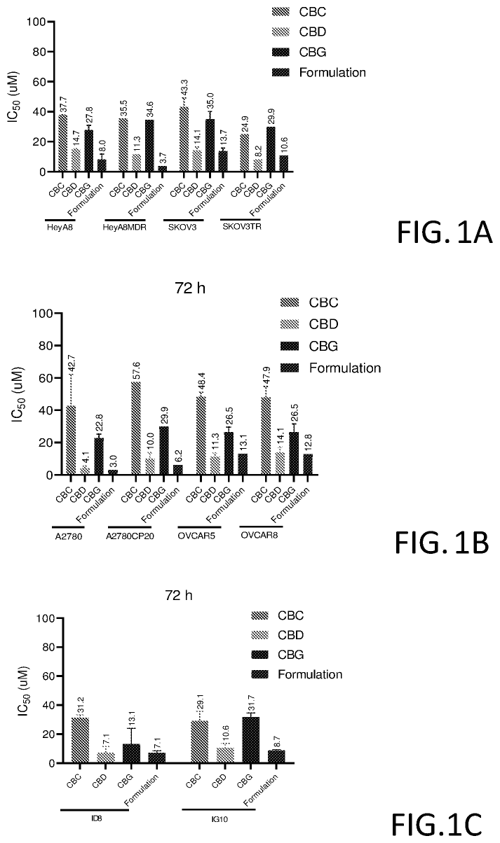

Cell viability assays were performed by testing ovarian cancer cells' metabolic ability to reduce the tetrazolium salt 3-(4,5-dimethylthiazol-2-yl)-5-(3-carboxymethoxyphenyl)-2-(4-sulfophenyl)-2H-tetrazolium (inner salt) to a formazan. To determine the cytotoxicity of the cannabinoids and annelix, cells were seeded in a 96-well plate and treated for 24, 48 and 72 h with either single agent cannabinoid or annelix at increasing concentrations between 0 and 100 micromolar based on CBD concentration in annelix. Activities for each reagent were determined using Hill plot analyses (Graphpad Prism 8 software) to assess the half maximal effect—IC₅₀. Results. In vitro screening of single agent cannabinoids CBD, CBC and CBG and annelix in 10 different ovarian cancer cell lines, including drug resistant and drug sensitive clones, showed that the multi cannabinoid terpenoid fixed dose formulation annelix was more potent compared to single agent CBG or CBC, having similar IC₅₀ values between 17-50 μM. Among the three cannabinoids, CBD was more potent than CBG or CBC with IC₅₀ values ranging from 7.8-14.7 M. In 4 (HeyA8, HeyA8-MDR, A2780, A2780CP20) of the 10 ovarian cancer cell lines, annelix was significantly more potent than CBD alone (p<0.05), while reduced or equal potencies were found in the remaining 6 cell lines as shown in FIG. 1, which illustrates the in vitro anti-cancer activity of single agent cannabinoids and annelix following 72 hour incubations. This led to the conclusion that the annelix formulation, when compared to CBD alone, has equal or improved anti-tumor effects in ovarian cancer cell lines, independent of drug sensitivity status and BRCA gene status. From FIG. 1, it is seen that the annelix formulation preferred embodiment drug product showed the highest potency (IC₅₀ 2.9-12.8 μM); that the potency of the single cannabinoid agents was in order as CBD>CBG>CBC; that CBD's potencies were IC₅₀ 7.8 to 14.7 μM; CBC's potencies were IC₅₀ 25.9 to 49.9 μM; and that CBG's potencies were IC₅₀ 17 to 35 μM; and that activity was shown against drug sensitive and multi-drug resistant cell lines. It is presently believed that the mechanism of action for the annelix formulation is to downregulate cell cycle and DNA damage checkpoints (i.e. ATM Wee1, CDC25, Rad51, chk1. ATR, ATM, and p53); and to upregulate Her-2 signaling in breast cancer and Pi3K.

### Example 7

The purpose of this experiment was to assess the activity of a formulation produced from single agents of key cannabinoids and terpenes in DMSO (in the same ratios as for aqueous annelix) and compare the resultant activity to annelix (aq.). Single agents (CBD, CBC, CBG, bisabolol, α-terpinene, and camphene) were obtained from Restorative Botanicals company as purified substances. All compounds were dissolved in DMSO except for camphene, which was dissolved in ethanol. Aliquots of each of the stock solutions were used to produce the same ratio of key components as is annelix (i.e. α-terpinene 0.22 M, bisabolol 0.21 M, camphene 0.22 M, CBC 0.06 M, CBD 0.3 M, and CBG 0.06 M).

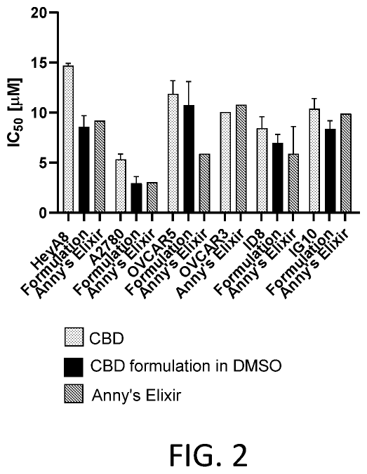

The anti-cancer activity for each comparator group was determined as described in the method under Experiment 6. Cells were incubated for 72 hours after which time cell viability assays were performed and the activities calculated as IC₅₀ values. Results. Anti-cancer activities for the alternative embodiment cannabinoid/terpene formulation in DMSO, compared to annelix (aq.), are illustrated in FIG. 2, and showed similar activities in all tested ovarian cancer cell lines between 2.9-13.7 μM. In 3 of 6 cell lines; the tested annelix in DMSO was more potent in killing ovarian cancer cells than CBD alone. In OVCAR5, the cannabinoid/terpene DMSO formulation was less potent compared to annelix (aq.). This led to the conclusion that annelix (aq.) has increased anti-cancer potency compared to isolated CBD, and as compared to annelix in DMSO.

### Example 8

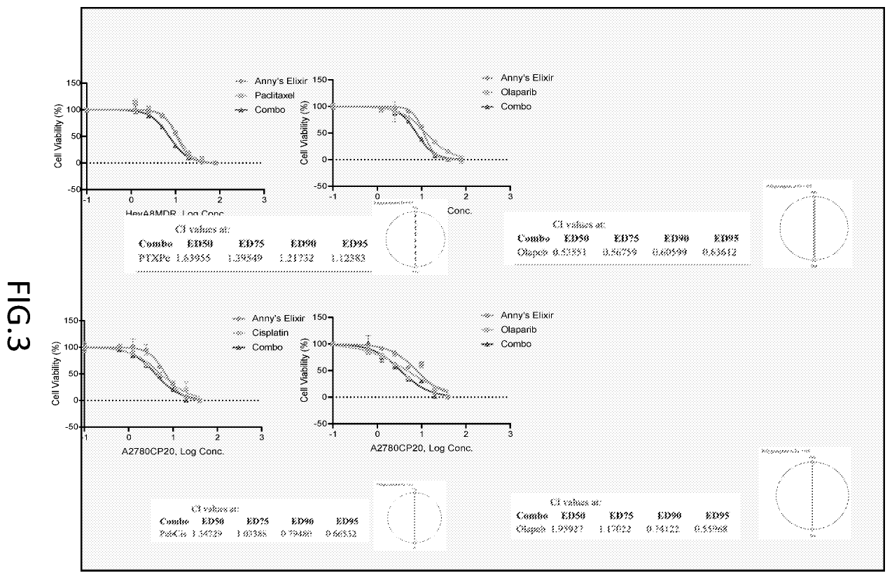

The purpose of this experiment was to determine whether annelix (aq.) synergizes with standard of care cancer therapeutics. Annelix (aq.) was combined with paclitaxel, cisplatinum and the PARP inhibitor olaparib. The drug resistant ovarian cancer cell lines HeyA8MDR, and A2780CP20 were tested. Both cell lines have a BRCA wildtype gene signature and are resistant to PARP inhibitors. Cells were prepared as described in Experiment 6. Single agent paclitaxel, cisplatinum and olaparib were added to cell cultures in a serial dilution ranging from 0.1 to 80 μM. Annelix (aq.) was added corresponding to 0-80 μM based on CBD content. Combination studies were conducted using a constant ratio format for the incubations of annelix (aq.) and paclitaxel in HeyA8MDR, annelix (aq.) and cisplatinum in A2780CP20 and annelix (aq.) and olaparib in both cell lines were conducted. Cell viability was determined after 72 and 96 hours of incubation. Combination indices were assessed based on activities for single agents using the Compusyn software package. Results. As per the definition of the Combinational Index (CI), a value of <0.1 indicates very strong synergism; values of 0.10-0.30 indicate strong synergism; values of 0.31-0.70 indicate synergism; values of 0.71-0.85 indicate moderate synergism; values of 0.86-0.90 indicate slight synergism; values of 0.91-1.10 indicate being nearly additive; values of 1.11-1.20 indicate slight antagonism; values of 1.21-1.45 indicate moderate antagonism; values of 1.46-3.30 indicate antagonism; values of 3.31-10 indicate strong antagonism; and values of >10 indicate very strong antagonism. The combination of annelix (aq.) and cisplatinum was synergistic with a combination index of 0.6-0.8. The combination of annelix (aq.) and olaparib was synergistic in both BRCA WT cell lines with combination indices of 0.6-0.75 for A2780CP20 and 0.53-0.63 for HeyA8MDR (FIG. 3). The combination with annelix (aq.) and paclitaxel was slightly additive in HeyA8MDR, with a combination index of 1.12-1.6 as illustrated in FIG. 3, which illustrates Hill plots of incubations for four selected single agent standard of care drugs, annelix (aq.), and the combinations of annelix (aq.) with each selected drug. Hey8MDR cells were incubated for 72 hours, A2780CP20 cells were incubated for the combination of annelix (aq.) and olaparib was for 72 hours, and A2780CP20 cells in combination with annelix and cisplatinum were incubated for 96 hours. Results from Compusyn software analysis for the combination indices are listed below each plot in FIG. 3. The polygonogram for each plot depicts the calculated assessment for synergy and slight additive effects. Conclusion. Annelix (aq.) is synergistic in drug resistant ovarian cancer cell lines. The strongest synergy was found for the combinations of annelix (aq.) with olaparib and for annelix (aq.) with cisplatinum. Based on these results, annelix (aq.) may improve responses in drug resistant ovarian cancer.

### Example 9

### In Vivo Dose Finding and Efficacy Study.

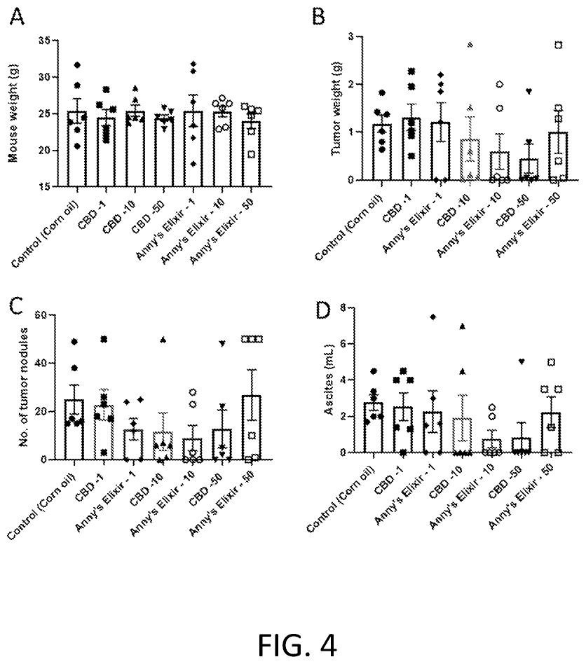

The purpose of this study was to determine the safety and efficacy of single agent CBD and annelix in an orthotopic xenograft model for ovarian cancer. This mouse model resembles the disease of ovarian cancer in humans with development of tumor nodules in the abdominal cavity and development of ascites. Method. Luciferase transfected human ovarian tumor cells, OVCAR5 Luc (1.0 million) were injected into the peritoneal cavity of nude mice, intra peritoneally (i.p.). On day 8 after tumor cell injection IVIS® optical imaging was performed to confirm tumor establishment. Mice that had established tumors were randomized into treatment groups of 6 mice each and treated 3 times per week until day 32 after tumor cell injection with CBD dissolved in DMSO or annelix (aq.) administered at a volume of 100 μL into the peritoneal cavity. Body weights were monitered and mice were observed for any adverse events. The study was an approved protocol of the MD Anderson Cancer Center (MDACC) Institutional Animal Care and Use Committee (IACUC). Treatment groups were as follows: Controls (corn oil), 1, 10 and 50 mg/kg CBD, 1, 10 and 50 mg/kg, and annelix, where dosage of annelix per kg of body weight was based on annelix's CBD content. Mice were sacrificed on day 32 (post inoculation of cells) and tumors were harvested. Body weights, tumor weight, number of tumor nodules and ascites volumes were recorded. Results. The body weights, tumor weights, tumor nodules and ascites volumes for each of the treatment groups are shown in FIG. 4. FIG. 4 illustrates a safety and efficacy study in an OVCAR5Luc orthotopic xenograft model after intraperitoneal injection of CBD or annelix (aq.) at doses of 1, 10 and 50 mg/kg based on CBD contents. Control mice received corn oil injections. Body weights were not affected by either the dose of annelix (aq.), nor by the dose of CBD in this study. FIG. 4A illustrates that both treatment regimens were well tolerated and did not cause any adverse effects. Mice treated with CBD (50 mg/kg) and annelix (aq.) (10 mg/kg) showed reduction in tumor burden compared to control group (FIG. 4 B). Tumor weight control by annelix (aq.) (10 mg/kg) was superior compared to single agent CBD (10 mg/kg) (FIG. 4 B). Tumor nodules control was improved over CBD alone at a dose of 1 mg/kg for annelix (aq.) (FIG. 4 C). Ascites volume was controlled by annelix (aq.) at a dose level of 10 mg/kg compared to single agent CBD at a dose level of 50 mg/kg (FIG. 4 D). Statistical significance was not reached. Conclusion. Annelix (aq.) demonstrated safety at doses of up to 50 mg/kg and controlled tumor weights, tumor nodules and ascites at lower dose levels when compared to single agent CBD. Annelix (aq.) may therefore be suitable as a palliative agent for the side effects of chemotherapy in patients with ovarian cancer as it clearly does not promote tumor growth.

### Example 10

The purpose of this experiment was to assess changes in protein expression levels elicited by either annelix (aq.) or CBD alone as well as possible mechanisms of annelix-induced cytotoxic effects. Two ovarian cancer cell lines (A2780 and OVCAR5) were treated with DMSO, CBD (5 μM), or annelix (based on CBD content of 5 μM) for 8 and 24 hours and performed Reverse Phase Protein Array (RPPA) and Ingenuity Pathway Analysis (IPA). Results. RPPA results showed that by comparison, an 8 hour treatment was more effective than a 24 hour treatment. RPPA array showed that the top most affected pathways were hereditary breast cancer signaling, cell cycle and DNA damage checkpoint regulation. ATM signaling, and HER-2 signaling in breast cancer upon the treatment of cells with annelix (aq.) versus DMSO. The most highly affected pathways upon the treatment of cells with CBD versus DMSO were HER-2 signaling in breast cancer, PI3/AKT signaling, molecular mechanisms of cancer, systemic lupus erythematosus in B cell signaling pathway, and autophagy. The most upstream regulators are MYC, E2F1, EGF and TP53 for annelix (aq.) and p53, AKT, E2F1, beta-estradiol and ERBB2 for CBD. Cell cycle checkpoint and DNA damage repair pathway proteins including Wee1, CDC25, Rad51, chk1, ATR, ATM and p53 were downregulated upon the treatment of cells with annelix (aq.) when compared to DMSO control, whereas very few proteins, including ATR and CDC25, were downregulated in CBD-treated cells as compared to cells treated with annelix (aq.). Conclusion. Cells treated with annelix (aq.) inhibited several proteins that are involved in cell cycle checkpoint activity and a DNA damaging repair pathway, as compared to CBD-treated cells.

### Drug Product Development Candidate Examples.

### Example 11. Optimization and Characterization of a Novel Multi-Cannabinoid, Multi-Terpenoid Formulation to Inform Lead Drug Candidate Selection

A prototype MCTF, annelix, consists of a fixed dose combination of 3 cannabinoids, namely cannabidiol (CBD), cannabichromene, CBC, and cannabigerol, CBG) and 3 terpenes, alpha terpinene, bisabolol, and camphene. To simplify this cannabinoid/terpene formulation by reducing the number of compounds in the combination and then to optimize the cannabinoid:terpene ratio, in vitro and in vivo studies in ovarian cancer (OC) cells and models are conducted to select a lead drug development candidate that is equal or superior in efficacy to annelix. Ten formulations are prepared from botanical raw plant products containing various cannabinoid:terpene ratios. Two different terpenes are tested to determine which is more active when combined with cannabinoids. 10 candidate formulations are tested in initial in vitro studies and then in vivo studies in 4 human OC models and 1 murine OC model. Subsequent studies focus on the 3 most active formulations from the initial studies. The 3 most active formulations are tested in combination with standard of care (SOC) OC therapeutics (cisplatinum, olaparib, paclitaxel, vincristine, gemcitabine, doxorubicin) in vitro using the multi-drug resistant OC cell lines HeyA8MDR and A2780CP20. Combination studies are conducted in constant ratio format to assess degree of synergy of the MCTF formulations with each SOC therapeutic tested. To further investigate safety and anti-tumor efficacy of the 3 most potent MCTF formulations, single agent studies are conducted using 1 and 10 mg/kg doses in an orthotopic syngeneic murine model of OC (ID8.luc) with a BRCA wildtype gene signature. Treatments are administered 3 times weekly for 4 weeks, with tumor burden, number of tumor nodules and ascites fluids subsequently determined. Body weights are monitored as an index of MCTF safety. The MCTF formulation with the highest single-agent efficacy is used for a combination study with cisplatin, a PARP inhibitor and a checkpoint inhibitor (anti-PDL1) in the ID8.luc model. From mice treated with CPI, immune profiling is conducted from tumors using FACs analysis to determine the effect of the MCTF formulation on immune effector and suppressor cell populations utilizing murine markers for CD4+ T cells. CD8+ T cells, regulatory T cells. MDSC, gMDSC, mMDSC, tumor associated macrophages. B and NK cells. Relying on earlier studies that showed that annelix downregulated proteins involved in DNA repair and cell cycle signaling, confirmatory and extension of prior MOA analyses is performed to assess the effect of the 3 lead formulations as a single agent, and in combination with SOC drugs olaparib and cisplatin. These studies will help to select the most potent MCTF for IND-enabling studies for development in OC patients in combination with SOC.

### Example 12. Optimization and Characterization of a Novel Multi-Cannabinoid, Multi-Terpenoid Formulation to Inform a Fixed-Dose Composition Formulation Research Program

A prototype first-in-class MCTF, annelix, consists of a fixed dose combination of 3 cannabinoids, namely cannabidiol (CBD), cannabichromene, CBC, and cannabigerol. CBG) and 3 terpenes, alpha terpinene, bisabolol, and camphene. A compounding sequence is used in order to research the effect of altering composition component deletions, substitutions, or additions to the fixed dose combinations of this cannabinoid/terpene strategic approach. e.g. annelix, by: (a) formulating various combinations of the compound types. (b) determining the number of compounds in the resultant combination, and then to (c) optimize the cannabinoid:terpene ratio, in vitro and in vivo studies in ovarian cancer (OC) cells and models are conducted to select formulation program leads that may progress into drug development candidates that are equal or superior in efficacy to annelix. Formulations are prepared from botanical raw plant products containing various cannabinoid:terpene ratios. In the compounding sequence, each different terpene is tested to determine which is more active when combined with each other terpene and each cannabinoid. Conversely, each different cannabinoid is tested to determine which is more active when combined with each other cannabinoid and each other terpene. Thus, to exemplify this compounding sequence, cannabidiol is tested in compounded combination with (1) cannabichromene; then with (2) cannabichromene and cannabigerol; then with (3) cannabichromene and cannabigerol and α-terpinene, and so on through all five possible combinations using the formulation preparation methods taught above. The same compounding sequence is then used for each preferred compound in all possible combinations of the rest of the six preferred compounds. Alternative cannabinoids or terpenes are substituted for any given preferred compounds as desired and the compounding sequence is then conducted. The compounding sequence is likewise adapted for all possible combinations of seven, or more, preferred compounds such as botanicals, e.g. polysaccharide K or other extractives of T. versicolor, or novel compounds or compounds new to the testing program. The resulting all possible multiple composition fixed dose candidate formulations are tested in initial in vitro studies and then in vivo studies by using the method of Example 11 above.

A Pre-Investigational New Drug (IND) package will contain all available preclinical data on safety and efficacy for the lead MCTF that is selected, obtained in vitro and in vivo in combination with SOC and CPI, initial toxicity profile and MOA data. Also included will be study designs for IND-enabling toxicity studies in 2 species. PK assessments, protocols for manufacturing and development for chemistry, manufacturing and control (CMC) processes, specifications for the MCTF and a description of the formulation. The clinical development plan incorporates design of a Phase I study in healthy volunteers and future planned Phase II/III studies in OC patients using an MCTF as palliative treatment during SOC therapy. A list of questions will be presented to the FDA staff intended to assess the sufficiency of planned IND-enabling studies for successful IND submission/approval. The studies proposed below are modified based on the pre-IND meeting FDA staff advice received. Pre-IND package preparation will be assisted by Cancer Insight, a Texas CRO.

### Example 13. cGMP Manufacturing of a MCTF for Clinical Studies

CBD is presently a Schedule I Controlled Substance compound under the federal Controlled Substances Act that requires development as a regulated drug. As with annelix, the cannabinoids in a new formulation will contain less than 0.3% THC on a dry weight basis to effectively eliminate psychoactive activity of the drug. Certificates of Analysis (COA) for each ingredient and for a final MCTF formulation are produced in compliance with FDA requirements. For each of the raw botanical components, all applicable chemistry, manufacturing and controls analyses and Botanical Raw Material documents are prepared. These documents will also be part of an Investigational New Drug (IND) application package for submission to the FDA. Botanical Raw Material release documentation from a hemp supplier is summarized and made available for inclusion in an IND submission. Drug Master Files (DMF) of the hemp manufacturer and a letter of authorization will be provided to the FDA. The three APIs of CBD, CBC, and CBG, and additionally terpene, will be in the form of isolated botanical extracts from the flowers of the Cannabis sativa L. plant prepared under cGMP conditions. For each of the APIs, all

Formulation of the new MCTF is conducted by a suitable hemp formulation contract formulation company, for example Hemp Depot (Colorado Springs, CO), or Restorative Botanicals (Longmont, CO). APIs are dissolved in MCT oil to produce a drug product in a non-sterile, non-preserved, non-aqueous oral solution. The formulation is manufactured to meet specifications on molecular weight, purity, residual solvents, heavy metals, water, bioburden and bacterial endotoxin, and viscosity in the final product. Stability is determined for the drug product in solution, including breakdown product analysis. A manufacturing batch of 500 g of the MCTF based on CBD content is prepared, which is projected to be sufficient to complete pre-clinical acute and repeat toxicology studies in 2 animal species, as well as a Phase I clinical study in healthy human subjects. This amount is based on once daily dosing for up to 4 cohorts with 3 patients in a clinical study in healthy human subjects receiving oral formulation for up to 56 days. IND-enabling toxicology studies are conducted with the MCTF in vitro to assess inhibition/induction of cytochrome P450 isoenzymes and drug-drug interactions with standard of care therapeutics. Good Laboratory Practice (GLP) toxicology studies in 2 animal species are performed for acute and repeat dose toxicity, followed by pharmacokinetic analyses.

### Example 14 Acute (14 Day) and Repeat Dose Toxicity (28 Days) Study in Mice

A certified Contract Research Organization (CRO), for example Stillmeadow, Inc., a Texas based CRO, is employed for this study. A 14-day, single dose GLP acute dose study in mice (N=20) is conducted with 3 different dosages of CBD formulation (low, mid, and high). A PK/PD analysis is conducted in a subset of animals. A second toxicology study, a pivotal study, includes 28-day GLP repeat-dose toxicology studies. Study results and summaries are included in an IND submission. The cannabinoid/terpenoid formulation are prepared from the botanical raw materials and administered via oral ingestion.

### Example 15 Acute (14 Day) and Repeat Dose Toxicology (28 Days) in Dogs

A suitable certified CRO, for example Stillmeadow, Inc. is employed for this study. A 14-day, single dose GLP acute study in dogs (N=6) is conducted with 3 different doses of MCTF (low, middle, and high). A PK/PD analysis is conducted in a subset of animals. A second toxicology study, which is a pivotal study, will include 28-day GLP repeat-dose toxicology studies. Study results and summaries will be included in an IND submission to the FDA. The cannabinoid/terpenoid formulation will be prepared from the botanical raw materials and administered via oral ingestion.

### Example 16. Development and Validation of a Method to Determine Serum Cannabinoid Levels

A suitable CRO, for example Pyxant Labs, Colorado Springs, Colorado, is employed to develop extraction and detection methods for CBD, CBC, CBG, and terpenes from fresh blood. Detection includes liquid-liquid extraction and separation of cannabinoids, terpenes and metabolites by HPLC chromatography. An assay is developed and validated, defining the CBD detection range (expected to be in a range of 1-1000 ng/ml) for serum of 3 species (mice, dogs, human). The validated method (GLP) will then be used for PK determinations in a Phase I clinical trial. Whole blood from mice, dogs, and humans is used for in vitro PK studies. Stability of cannabinoids is assessed using plasma and blood obtained from relevant species. Samples are incubated at 37° C. with a range of cannabinoids concentrations at timepoints spanning 0.25, to 192 hours. After extraction, cannabinoids and metabolite concentrations within the plasma samples are quantified at each time point by HPLC.

### Example 17. Pharmacokinetics in Mice and Dogs

The nonclinical pharmacokinetics and metabolism of cannabinoids is evaluated under non-GLP conditions in both in vivo and in vitro studies using the validated protocol. In vivo studies will be conducted using mice and dogs for PK, toxicology, and toxicokinetics (TK) in single dose studies. The oral route of administration will be used for in vivo studies, as this is the intended clinical route of drug administration. Cannabinoids are assessed based on the assay validated for each of the two species employed in the toxicology studies, above. Blood samples will be drawn at the following time points: −5, 0, 2, 5, 15, 30, 60, 120, 240, 480 mins. Concentration-time profiles will be plotted from plasma sample data. From the area under the plasma concentration time curve (AUC), slope of terminal decay phase PK parameters are calculated, including terminal half-life (T_{1/2}), total body clearance (CL), time of maximal concentration (Tmax), and volume of distribution (VD).

### Example 18. Absorption Studies

Detection of cannabinoids in the plasma of mice and dogs is determined following a single oral dose given at 3 different dosage levels (low, mid, high) comparable to doses that will be tested in clinical Phase I studies (50-300 mg/patient/day). Results of these studies will yield an estimate for terminal half-life of the cannabinoids. Cannabinoids are metabolized in the liver. In vitro studies will be conducted to determine MCTF induction of cytochrome P450 isoform (CYP) activity in freshly prepared human hepatocytes (Cyprotex, Watertown MA). Inhibition of CYP450 isoforms will be determined in human liver microsomes. The calculated half-life in incubations with human liver microsomes will determine the degree of metabolism of the cannabinoid formulation. Drug-drug interactions will be assessed in liver microsomes for combinations of cannabinoid formulation with cisplatin, olaparib, paclitaxel, vincristine, gemcitabine, and doxorubicin.

While the above description contains much specificity, these should not be construed as limitations on the scope of any embodiment, but as exemplifications of the presented embodiments thereof. Many other alternative embodiments and variations are possible within the teachings of the various embodiments. While the invention has been described with reference to exemplary embodiments, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention will not be limited to any particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are, unless otherwise stated, used in a generic and descriptive sense only and not for purposes of limitation, the scope of the invention therefore not being so limited. Moreover, the use of the terms first, second, etc. do not denote any order or hierarchy of importance, but rather the terms first, second, etc. are used to distinguish one element from another. Furthermore, the use of the terms a, an, etc. do not denote a limitation of quantity, but rather denote the presence of at least one of the referenced items.

While the invention has been described, exemplified, and illustrated in reference to certain preferred embodiments thereof, those skilled in the art will appreciate that various changes, modifications, and substitutions can be made therein without departing from the spirit and scope of the invention. It is intended, therefore that the invention be limited only by the scope of the claims which follow, and that such claims be interpreted as broadly as is reasonable.

What is claimed is:

1. A pharmaceutical drug formulation for administration to a patient in need thereof, comprising one or more cannabinols selected from the group consisting of:
(a) cannabidiol and the pharmaceutically acceptable salts, esters, solvates, optical isomers, and geometric isomers thereof, present in a molar concentration of from 0.03M to 3.0M;
(b) cannabigerol and the pharmaceutically acceptable salts, esters, solvates, optical isomers, and geometric isomers thereof, present in a molar concentration of from 0.006M to 0.6M; and
(c) cannabichromene and the pharmaceutically acceptable salts, esters, solvates, optical isomers, and geometric isomers thereof, present in a molar concentration of from 0.006M to 0.6M;
and further comprising one or more terpenes selected from the group consisting of:
(d) α-terpinene and the pharmaceutically acceptable salts, esters, solvates, optical isomers, and geometric isomers thereof, present in a molar concentration of from 0.022M to 2.2M;
(e) bisabolol and the pharmaceutically acceptable salts, esters, solvates, optical isomers, and geometric isomers thereof, present in a molar concentration of from 0.021M to 2.1M; and
(f) camphene and the pharmaceutically acceptable salts, esters, solvates, optical isomers, and geometric isomers thereof, present in a molar concentration of from 0.022M to 2.2M.

2. The pharmaceutical formulation as claimed in claim 1, wherein cannabidiol is present in a molar concentration of 0.3M.

3. The pharmaceutical formulation as claimed in claim 1, wherein cannabigerol is present in a molar concentration of 0.06M.

4. The pharmaceutical formulation as claimed in claim 1, wherein cannabichromene is present in a molar concentration of 0.06M.

5. The pharmaceutical formulation as claimed in claim 1, wherein α-terpinene is present in a molar concentration of 0.22M.

6. The pharmaceutical formulation as claimed in claim 1, wherein bisabolol is present in a molar concentration of 0.21M.

7. The pharmaceutical formulation as claimed in claim 1, wherein camphene is present in a molar concentration of 0.22M.

8. The pharmaceutical formulation as claimed in claim 1, additionally comprising one or more compositions selected from monk fruit extract present in a concentration ranging from 0.0025% to 0.25% w/w and ginger essential oil present in a concentration ranging from 0.059% to 5.9% w/w.

9. The pharmaceutical formulation as claimed in claim 8, wherein said monk fruit extract is present in a concentration of 0.025% w/w.

10. The pharmaceutical formulation as claimed in claim 8, where said ginger essential oil is present in a concentration of 0.59% w/w.

11. The pharmaceutical formulation as claimed in claim 1, additionally comprising polysaccharide K.

12. A method of treating an animal or human patient in need thereof to alleviate the adverse side effects of cancer chemotherapy, comprising the step of administering a pharmacologically effective amount of the pharmaceutical formulation as claimed in claim 1.

13. A method of manufacturing a pharmaceutical formulation comprising one or more cannabinols selected from the group consisting of: cannabidiol and the pharmaceutically acceptable salts, esters, solvates, optical isomers, and geometric isomers thereof, present in a molar concentration of from 0.03M to 3.0M; cannabigerol and the pharmaceutically acceptable salts, esters, solvates, optical isomers, and geometric isomers thereof, present in a molar concentration of from 0.006M to 0.6M; and cannabichromene and the pharmaceutically acceptable salts, esters, solvates, optical isomers, and geometric isomers thereof, present in a molar concentration of from 0.006M to 0.6M; and further comprising one or more terpenes selected from the group consisting of: α-terpinene and the pharmaceutically acceptable salts, esters, solvates, optical isomers, and geometric isomers thereof, present in a molar concentration of from 0.022M to 2.2M; bisabolol and the pharmaceutically acceptable salts, esters, solvates, optical isomers, and geometric isomers thereof, present in a molar concentration of from 0.021M to 2.1M; and camphene and the pharmaceutically acceptable salts, esters, solvates, optical isomers, and geometric isomers thereof, present in a molar concentration of from 0.022M to 2.2M, said method of manufacturing comprising the steps of:
(a) combining at least one medium chain triglyceride oil with full spectrum hemp distillate, one or more cannabinols selected from cannabigerol isolate, and cannabichromene distillate in a cooking vessel;
(b) heating the combined compositions of step (a) until a temperature of from 140 degrees F. to 200 degrees F. has been reached;
(c) cooling the product of step (b) to room temperature; and
(d) adding said one or more terpenes and mixing until homogenized.

14. The method of claim 13, comprising the additional step of adding monk fruit extract to said heated mixture before said cooling begins.

15. The method of claim 13, comprising the additional step of adding ginger essential oil to said cooled mixture.

16. The formulation as claimed in claim 1, further comprising one or more terpenes selected from the group comprising alpha-pinene, beta-pinene, camphene, beta-myrcene, D-limonene, 1,8-cineol, alpha-terpinene, gamma-terpinene, cis-beta-terpineol, terpinolene, linalool, fenchol, borneol, alpha-terpineol, alpha-cubebene, ylangene, alpha-copaene, beta-caryophyllene, alpha-bergamotene, alpha-humulene, alloaromadendrene, alpha-amorphene, (−)-lepidozene, beta-selinene, beta-cadinene, alpha-selinene, beta-dihydroagarofuran, alpha-bisabolol, beta-bisabolene, valencene, epizonarene, the selina-dienes, (−)-caryophyliene oxide, guaiol, 8-epi-gammaeudesmol, gamma-eudesmal, agarospiral, alpha-eudesimol, alpha-gurjunene, beta-eudesmaol, alpha-eudesmol, bulnesol, (−)-anymol, delta3-carene, alpha-ocimene, beta-ocimene, trans-alpha-bergamotene, and juniper camphor.

17. The formulation as claimed in claim 1, further comprising one or more cannabinoids selected from the group consisting of cannabigerol (CBG), cannabidiol (CBD), cannabichromene (CBC), cannabigerivarin (CBGV), cannabidivarin (CBDV), and cannabichromevarin (CBCV).

18. A method of treating a patient in need thereof, comprising the steps of administering to said patient a pharmacologically sufficient dose of a pharmaceutical drug formulation comprising one or more cannabinols selected from the group consisting of:
(a) cannabidiol and the pharmaceutically acceptable salts, esters, solvates, optical isomers, and geometric isomers thereof, present in a molar concentration of from 0.03M to 3.0M;
(b) cannabigerol and the pharmaceutically acceptable salts, esters, solvates, optical isomers, and geometric isomers thereof, present in a molar concentration of from 0.006M to 0.6M; and
(c) cannabichromene and the pharmaceutically acceptable salts, esters, solvates, optical isomers, and geometric isomers thereof, present in a molar concentration of from 0.006M to 0.6M;
and further comprising one or more terpenes selected from the group consisting of:
(d) α-terpinene and the pharmaceutically acceptable salts, esters, solvates, optical isomers, and geometric isomers thereof, present in a molar concentration of from 0.022M to 2.2M;
(e) bisabolol and the pharmaceutically acceptable salts, esters, solvates, optical isomers, and geometric isomers thereof, present in a molar concentration of from 0.021M to 2.1M; and
(f) camphene and the pharmaceutically acceptable salts, esters, solvates, optical isomers, and geometric isomers thereof, present in a molar concentration of from 0.022M to 2.2M.

19. The method as claimed in claim 18, wherein said patient is being treated for a disease selected from the group consisting of cancer therapeutic-induced gastrointestinal adverse effects, cancer therapeutic-induced peripheral neuropathy, drug-resistant cancer, or drug-sensitive cancer.

20. The method as claimed in claim 19, wherein said drug-resistant cancer is ovarian cancer.
